(19) United States
(12) Patent Application Publication
(10) Pub. No.: US 2024/0041649 A1
(43) Pub. Date: Feb. 8, 2024

(54) OPTIMIZED CONCAVE MIRROR FOR HORIZONTAL EYE SPRAY APPARATUS WITH ON-AXIS LIGHT GUIDED USER ALIGNMENT

(71) Applicant: TWENTY TWENTY THERAPEUTICS LLC, South San Francisco, CA (US)

(72) Inventors: Timothy Stowe, Alameda, CA (US); Oleg Rumyantsev, Menlo Park, CA (US)

(21) Appl. No.: 18/230,134

(22) Filed: Aug. 3, 2023

Related U.S. Application Data
(60) Provisional application No. 63/394,645, filed on Aug. 3, 2022.

(51) Int. Cl. A61F 9/00

(52) U.S. Cl. CPC A61F 9/0026

(57) ABSTRACT

An eye sprayer includes a spray nozzle having a nozzle axis. the spray nozzle is configured to produce eye spray. The eye sprayer also includes a concave mirror with an opening configured to allow for the delivery eye spray from the spray nozzle. The eye sprayer further includes a light emitter configured to produce a visible light beam directed from the mirror along the nozzle axis.

### RELATED APPLICATION

This application claims the benefit of U.S. Provisional Application Ser. No. 63/394,645, filed on Aug. 3, 2022, the disclosure of which is hereby incorporated by reference herein in its entirety.

### TECHNICAL FIELD

This disclosure relates to an eye spray apparatus for the self-application of a mist or drops to a user's eye. The apparatus includes a concave mirror for viewing the eye and on-axis, light-guided user alignment features.

### BACKGROUND

Aligning an eyedropper with the eye can be very difficult and challenging. This is typically done on a trial-and-error basis, with the user holding the dropper over the eye, applying the drops, and adjusting position when the drop misses the target. While this might be acceptable for some eyedropper applications, e.g., saline solution or other over-the-counter eye treatments for dry eyes, red eyes, etc., it can be unacceptable for medications, which can be expensive and can require a precise application, e.g., at the center of the eye.

Some solutions for aligning eyedroppers with the eyes have been developed. For example, some eyedroppers include added mirrors that allow the user to view his/her eye while applying the drops. This solution, however, is not as simple as it would seem, as enabling the user to view the dropper while, at the same time, positioning it properly relative to the eye, is complicated. For many of these devices, the addition of the mirror requires the view being off-axis with the dropper nozzle so that the user's view of the mirror is not blocked by the nozzle. As a result, the user is required to exercise some degree of estimation or approximation in directing the drops to the desired location on the eye. As a result, despite the addition of the mirror, alignment issues persist and alignment remains difficult for the user. In fact, one might argue that using the mirror is more complicated and error-prone than simply viewing the nozzle directly.

Existing eyedropper designs are largely gravitationally based with mirror arrangements that require the user to estimate angles and distances to understand where the eye drop will actually land. Additionally, these mirrors tend to be flat, so older users with presbyopia, having a very far (>50 mm) near point in their focal range, the image will still be blurry and out of focus during use.

Implementing a curved mirror to provide some focus magnification and improved details is possible. Mirrors of this sort do, however, tend to be large. For older patients who must wear scleral contact lenses due to corneal abnormalities, accommodation gets weak as they age and they may need higher curvature mirrors with high magnification in close range. With the large curvature of mirror image devices, the virtual image of the eye is magnified and can be extended further out to allow for patients with presbyopia to see their own eyes in clear focus. While the image of the eye is in focus it is not optimized for both nearsighted and farsighted cases and is not optimized for use with an eyedropper. Thus there is a question of what curved mirror focal length is best for a wide variety of near and far sided users while, at the same time, allowing the device to be positioned close enough to effectively apply the eyedropper dose.

An additional problem with these devices lies with the fact that they are traditionally edge lit with light rings that extend around the periphery of the mirror. For a dropper device that must be positioned close to the eye, there needs to be some central illumination. As the device moves closer and closer to the eye surface, however, the peripheral ring lighting is working more and more at an angle, so its effectiveness is increasingly reduced. For a horizontal eye sprayer, the optimal distance between the mirror and the surface of the eye is only about 20-30 mm. As the mirror gets closer and closer to the eye, higher and higher power LEDs are required to provide the necessary degree of illumination from the shallow angle between the light ring and the target.

Horizontal eye sprayers can place the tip of the eye spray nozzle at the center of a concave mirror. This configuration aids in on-axis alignment of the nozzle with the eye. An example of this arrangement is shown in the now expired patent U.S. Pat. No. 3,640,274 A (1969). This arrangement can, however, be problematic. Configuring the nozzle to protrude from the center of the mirror leads to the possibility of error. If the nozzle axis of alignment and mirror optical axis of alignment are off by even a few degrees (e.g.) ±4°, the drops can miss the target. This is especially true in the vertical direction, as the eyelids are typically open only about −7 mm. Therefore, care must be exercised to ensure that the optical axis and the spray axis are coincident and parallel. Additionally, it is better from a reliability standpoint to have the nozzle to be positioned behind, and discharge through, a hole in the mirror. This way, the nozzle can be at least partially sheltered from outside dust or debris.

### SUMMARY

An eye sprayer includes a spray nozzle having a nozzle axis. the spray nozzle is configured to produce eye spray. The eye sprayer also includes a concave mirror with an opening configured to allow for the delivery eye spray from the spray nozzle. The eye sprayer further includes a light emitter configured to produce a visible light beam directed from the mirror along the nozzle axis.

According to one aspect, the opening in the mirror can include a series of slits, and the spray nozzle can include a series of nozzles. A nozzle can be positioned centrally in each slit. The light emitter can be configured to produce a light beam emitted through each slit.

According to another aspect, the eye spray can include at least one of drops, a mist, and a micro-sheet of eye drop fluid.

According to another aspect, a central axis of the mirror and the nozzle axis can be coincident and parallel.

According to another aspect, the light emitter can be is configured to direct the light through the spray nozzle along the nozzle axis.

According to another aspect, the eye sprayer can be configured to allow the user to view his/her eye at a prescribed working distance. The eye sprayer can also be configured to align the spray nozzle by aligning the image of their own eye with a view of the light source overlaid on top of it.

According to another aspect, the mirror can be configured to have a radius of curvature (ROC) configured to produce a degree of magnification that allows users with myopia or hyperopia/presbyopia to view his/her eye with the eye sprayer positioned at a prescribed working distance.

According to another aspect, the mirror can have a radius of curvature of 55±2 mm, a working distance of 25 mm. According to another aspect, the mirror can have a radius of curvature of 67±2 mm, a working distance of 30 mm.

According to another aspect, the mirror can be an interchangeable insert, and its radius of curvature can be chosen to allow optimal focus position based on the user eye prescription.

According to another aspect, the mirror can have a radius of curvature configured to allow the user to view his/her eye with sufficient clarity and lack of blurriness to allow them to align the view his/her eye with the eye sprayer positioned at a prescribed working distance.

According to another aspect, the mirror can be configured to accommodate users with myopia of at least −6D or hyperopia/presbyopia of at least +3D.

According to another aspect, the mirror can be semi-transparent and can allow viewing additional light sources located behind the mirror to aid alignment.

According to another aspect, the mirror can have a semi-transparency of about 30% transmission about 70% reflectivity.

According to another aspect, the light emitter can include a light ring concentric with the mirror and extending around the nozzle.

According to another aspect, the eye sprayer can also include a proximity sensor configured to determine the proximity of the sprayer to the eye and to produce a visual indication of the sprayer being inside or outside a prescribed range of working distances.

According to another aspect, the mirror can be semi-transparent and the proximity sensor can detect the proximity of the sprayer to the eye through the mirror.

According to another aspect, the proximity sensor can include an ultrasonic proximity sensor, a light-based position sensor, or a camera-based position sensor.

According to another aspect, the opening in the mirror can include a series of slits, and the light emitter can be configured to produce a light beam emitted through each slit. The spray nozzle comprises a series of nozzles, with a nozzle being positioned centrally in each slit.

### DRAWINGS

FIG. 1 is a perspective view illustrating a horizontal eye spray apparatus according to an example configuration.

FIG. 2 is a front view of the horizontal eye spray apparatus.

FIG. 3 is a side view of the horizontal eye spray apparatus.

FIG. 4 is a schematic view of certain components of the horizontal eye spray apparatus.

FIG. 5A is an enlarged schematic view of a portion of the horizontal eye spray apparatus.

FIGS. 5B-5D illustrate the operation of certain features of the horizontal eye spray apparatus of FIG. 5A during use.

FIGS. 6A and 6B illustrate factors that can affecting the operation of the horizontal eye spray apparatus.

FIG. 7 illustrates an accommodation range for users of the horizontal eye spray apparatus.

### DESCRIPTION

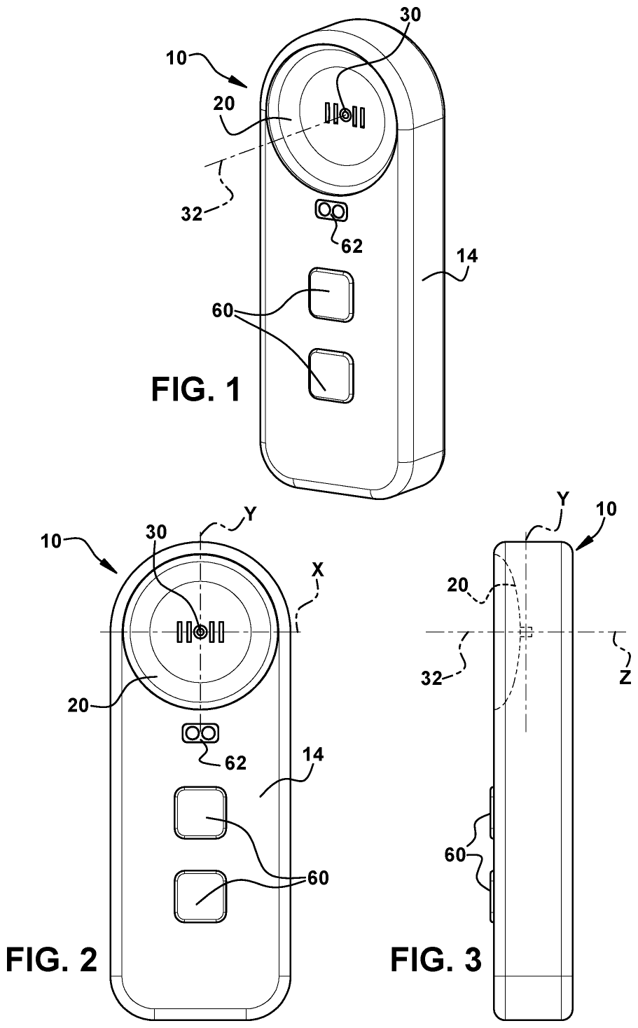

Referring to FIGS. 1-3, a horizontal eye spray apparatus or device 10 (“eye sprayer”) implements concave mirror 20 optimized for a horizontal eye spray nozzle 30 with illumination provided by one or more light sources 40, such as LEDs. The nozzle 30 is centrally located on the mirror 20 and is configured to direct a mist or spray along a central spray axis 32 that is centered on the mirror and extends perpendicularly or normal to a plane that is tangential to the center of the concave mirror. The LEDs 40 are arranged in a horizontal array on opposite sides of the spray axis 32 and are configured to aid in an alignment of the eye sprayer 10 with the eye so that the spray will be applied properly. This configuration allows the eye sprayer 10 to direct the mist or spray onto the eye in a repeatable and reliable manner, despite the fact that the angular requirements are very tight.

For example, if the nozzle is positioned about 25 mm away from the eye, and the drop diameter is 1.5 mm, there is only +/−2.0 mm of variation in aiming for the drop to hit the eye without hitting the eyelid. This translates into very precise angular requirements. To Further illustrate this point, assuming the eye spray nozzle 30 is perfectly concentric with the mirror 20, an angular deviation of only arctan( 2/30) or +/−4.5 degrees means even a perfectly centered image of the eye in the concave mirror would mean the edge of the drop would hit the eyelid. If the user has problems holding the dropper steady or the mirror isn't exactly concentric with the eye in terms of X-Y lateral displacements, the angular alignment requirements only get tighter. For any particular light path, any mechanical deviation due to mirror offsets in angle or X-Y displacement offsets can be corrected for by the user.

Proper alignment between the nozzle 30 and the eye is a function of two requirements: 1) that the nozzle(s) 30 is/are at the proper distance from the eye, and 2) that the nozzle(s) is/are aimed properly toward the target on the eye surface. To help achieve these requirements, the light source 40 is configured so that light from the individual LEDs passes directly through the nozzle 30 or in the area of the nozzle, in a manner such that the emitted light is aligned with the central spray axis 32. In this configuration, the geometry of the nozzle(s) 30 can be configured to limit the angular deviation of the light beam passing therethrough. By configuring the light source 40 to work in combination with distance/proximity sensing, the proper nozzle distance and dropper/spray trajectory can be achieved.

In the example configuration of the eye sprayer 10 shown in FIGS. 1-3, a housing 14 supports the concave mirror 20 and the nozzle 30, which is located centrally on the mirror and on the spray axis 32. The form factor of the eye sprayer 10 is by way of example only. Alternative designs could be implemented.

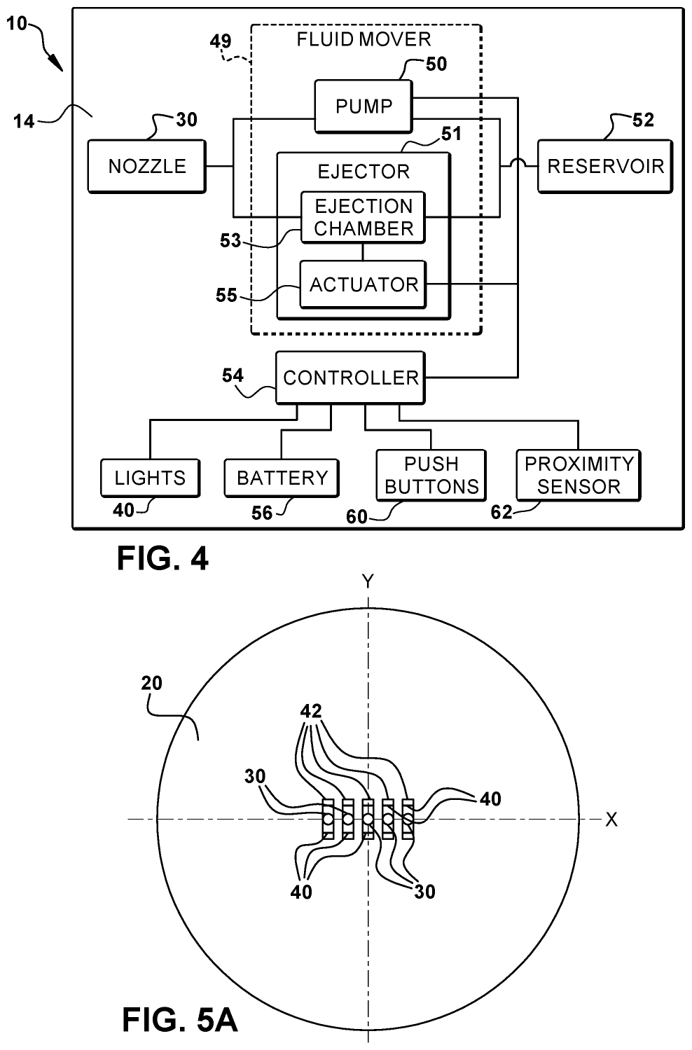

The mirror is configured to extend across a plane defined by an X-Y axis of the eye sprayer, with the nozzle axis 32 pointing perpendicularly along a Z-axis. The nozzle 30 can be configured to deliver eye spray fluids in the form of drops, mist, micro-sheet, or any other form. Although a single nozzle 30 is shown in FIGS. 1-3, the eye sprayer 10 could include multiple nozzles (see, e.g., FIG. 4). The nozzle 30 can be positioned flush with the surface of the mirror 20, rearward of the mirror surface, or can project from the mirror surface. A flush or rearward position relative to the mirror 20 might be preferred to help protect the nozzle 30 and keep it clean and free from debris.

Components internal to the eye sprayer 50 are illustrated schematically in FIG. 4. As shown in FIG. 4, the nozzle 30 is fluidly connected to a fluid mover 49 that delivers the fluid from a reservoir 52 to the nozzle(s) 30. The fluid mover 49 can be a pump 50 or an ejector 51. The reservoir 52 can be a disposable cartridge or a re-fillable chamber. The nozzle(s) 30 can be configured to produce a spray, drops, etc. depending on the application. Operation of the fluid mover 49 can be controlled by a controller 54, actuated by one or more pushbuttons 60 to supply battery power 56 to the fluid mover. The controller 54 can also control operation of the light sources 40 and a proximity sensor 62.

A series of light openings 42 in the mirror 20, in the form of an array of spaced slits, are positioned adjacent to and on opposite sides of the nozzle 30, with the illumination sources 40 positioned behind. Other shapes, configurations, and arrangements of light openings can be implemented. The pushbuttons 60 can control the operation of the light source 40 and the fluid mover 49.

In one example configuration, the fluid mover 49 can be a pump 50. In this configuration, the pump 50 can be controlled electronically by the controller 54, which supplies power to the pump from the battery 56 in response to inputs from the pushbuttons 60. In an alternative configuration, the pump 50 can be manually operated and the pushbutton 60 for operating the pump is a manually operated mechanical input, such as a trigger. This alternative configuration would be similar to a standard spray bottle configuration.

In another alternative configuration, the fluid mover 49 can be an ejector 51, including an ejection chamber 53 and an actuator 55. In one example configuration, the actuator 55 can be an electromagnetic actuator such as a bi-stable solenoid actuator. The actuator 55 actuates (e.g., pushes) a deformable membrane attached to or formed as a component of the ejection chamber 53. When the actuator 55 is actuated, the membrane deflects and deforms, which causes the fluid to be expelled from the ejection chamber 53, out of the ejector 51 to the nozzle 30. In the example solenoid configuration, this allows for pushing fluid out of the nozzles 30 from the ejection chamber 53 when the solenoid 55 is activated, for example, to an extended state. After ejection, the ejector 51 can also be configured to draw fluid back into the ejection chamber 53, for example, with the membrane drawing new fluid from the reservoir 52 into the ejection chamber in response to the actuator/solenoid 55 returning to a retracted state.

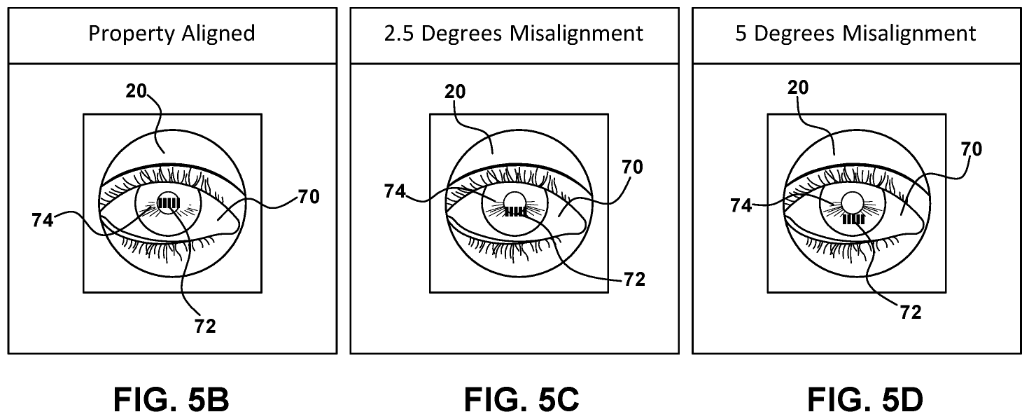

In the example configuration illustrated in FIGS. 1-3, the nozzle 30 is centrally located, with two slits 42 on either side allowing their associated illumination sources 40 to shine through. Another example configuration is illustrated in FIG. 5A. In the example configuration of FIG. 5A, one or more nozzles 30 can be provided in the slits 42 themselves, along with their associated light sources 40. The configuration of FIG. 4 provides an array of nozzles 30 that can produce an even spray in a highly controlled shape or pattern, such as an oval pattern. In this configuration, the illumination sources 40 coincide with the nozzles 30 so that the light beams they produce shines on-axis with the spray without any offset or parallax. In fact, this configuration can be especially advantageous in that it can allow the offset between the light beams and the spray axes of their associated nozzles 30 be zero. As a result of the nozzle axes coinciding with their corresponding light beam axes, the spray pattern produced by the by the eye sprayer 10 will be accurate and true to the light beams, so alignment based on the light beams, as described below, will produce an accurate spray application to the eye.

The light sources 40 can be configured to indicate proper positioning near/far distancing of the eye sprayer 10 relative to the eye. To do so, the eye sprayer 10 can include a proximity sensor 62 for sensing the distance between the sprayer and the user's eye. In this instance, the light sources can be configured to provide position feed back to the user. For example, red light can be used to indicate when the distance between the nozzle 30 and the eye is too far or too near, and green light can be used to indicate when the distance between the nozzle and eye is optimized for directing drops or spray from the nozzle into the eye.

In the example configuration of FIG. 5A, the light openings 42 can be configured to limit or control the direction in which light is emitted or passes through. The concave mirror 20 is configured to cover the area surrounding the light openings 42. The area covered by the mirror also covers the location where the eye spray nozzles 30 are located. The mirror 20 includes nozzle openings through which drops or a spray mist can be directed. These can, for example, be centrally located portions of the light openings 42 in the array arranged centrally in the concave mirror 20.

Referring to FIGS. 5B-5D, during operation of the eye sprayer 10, the user will see the image of their eye 70 on the mirror 20 as well as the light sources 40 shining through the light openings 42. This will appear to the user as light images 72 being superimposed onto the surface of the eye 70. Thus, during use, the user can judge whether the eye sprayer 10 is positioned the proper distance (e.g., via green lights) from the eye, and not too close/far (e.g., via red lights) through this reflection seen in the mirror 20.

Additionally and advantageously, the user can also judge whether the nozzles 30 are aimed correctly, that is, whether the nozzle axis/axes extend to the target location on the eye 70. Typically the target location will be the center of the eye 70, i.e., the center of the iris/pupil 74. To achieve proper alignment, the user manipulates the position and orientation of the eye sprayer 10 so that the light images 72 coincide with the iris/pupil 74. The light images 72 can be blurred due to being viewed at a close distance, but the image of the eye 70 itself can be magnified by the concave mirror 20 and can be in focus, depending, of course, on the user's vision.

FIGS. 5B-5D illustrate how the alignment of the light sources 40 with the iris/pupil 74 can be judged by the user. When alignment occurs, it is ensured that the nozzle axis 32 is pointing toward and extends through the iris/pupil 74. Alignment is achieved when the light images 72 are centered on and overlie the iris/pupil 74 as shown in FIG. 5A. FIGS. 5B and 5C show alignment errors of varying degrees. By utilizing the method described above, proper alignment using the device can result in alignment with in +/−2 degrees.

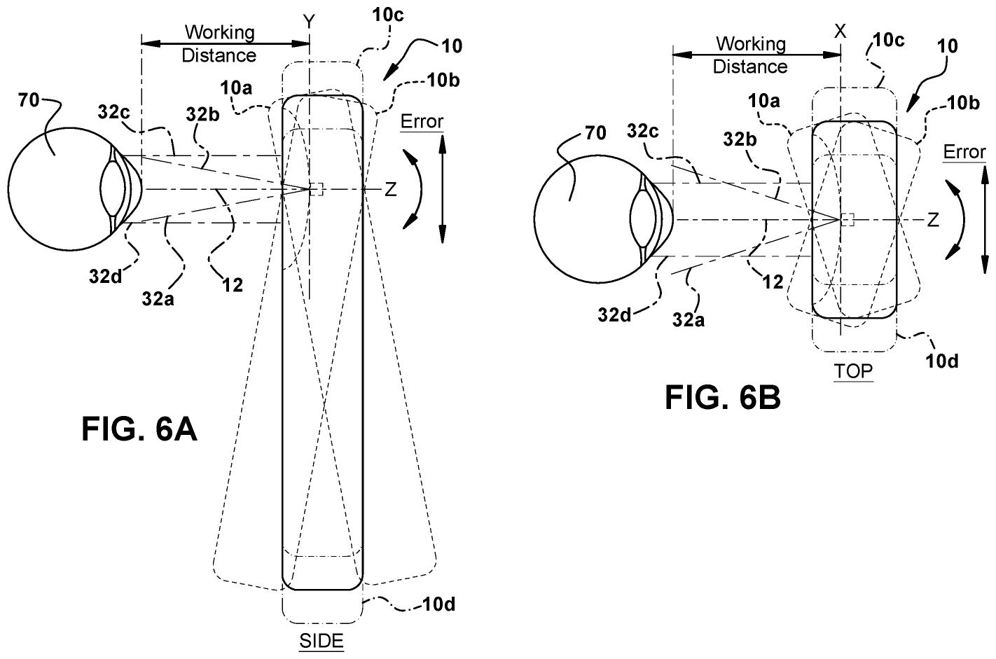

The misalignment scenarios shown in FIGS. 5B and 5C can be caused by a variety of errors, which are illustrated in FIGS. 6A and 6B. Referring to FIG. 6A, errors can occur due to rotation about the X-axis or a mis-positioning in the X-Y plane (i.e., shifting along the Y-axis in FIG. 6A). More specifically, a forward (counterclockwise) rotation about the X-axis, indicated generally at 10a, results in the error in the nozzle axis shown at 32a. Similarly, a rearward (clockwise) rotation about the X-axis, indicated generally at 10b, results in the error in the nozzle axis shown at 32b. Also, an upward shift in the X-Y plane along the Y-axis, indicated generally at 10c, results in the error in the nozzle axis shown at 32c. Further, a downward shift in the X-Y plane along the Y-axis, indicated generally at 10d, results in the error in the nozzle axis shown at 32d.

Referring to FIG. 6B, errors can occur due to rotation about the Y-axis or a mis-positioning in the X-Y plane (i.e., shifting along the X-axis in FIG. 6B). More specifically, a left (counterclockwise) rotation about the Y-axis, indicated generally at 10a, results in the error in the nozzle axis shown at 32a. Similarly, a right (clockwise) rotation about the Y-axis, indicated generally at 10b, results in the error in the nozzle axis shown at 32b. Also, an left (upward in FIG. 6B) shift in the X-Y plane along the X-axis, indicated generally at 10c, results in the error in the nozzle axis shown at 32c. Further, a right (downward in FIG. 6B) shift in the X-Y plane along the X-axis, indicated generally at 10d, results in the error in the nozzle axis shown at 32d.

Of course, a combination of the errors shown in FIGS. 6A and 6B can exist simultaneously. In any instance, positional adjustments are implemented by the user to 1) align the light image 72 with the iris/pupil 74 as described above, and 2) to adjust the working distance of the eye sprayer 10 to the eye 70 obtain indication of the proper working distance, e.g., a green light.

### Alternative Configuration—Semi-Transparent Mirror

In an alternative configuration of the eye sprayer 10, the mirror 20 is semi-transparent (e.g., 30% transmission 70% reflectivity) to allow for positioning the light sources 40 behind the mirror and transmission of the light through the mirror structure. This can, for example, allow for the light source 40 to be an LED ring light source, concentric with the nozzle axis 32, that allows the user to overlay the image of the light ring with some features of their own eye image, such as edge of the iris or edge of the pupil. The utilization of a semi-transparent mirror can also lend compatibility with other sensors (light based position sensors, camera-based detectors, proximity sensors, etc.) so that the sensor detects the signal through the mirror itself, thus eliminating the need for light openings in the mirror.

### Accommodating the User's Eye

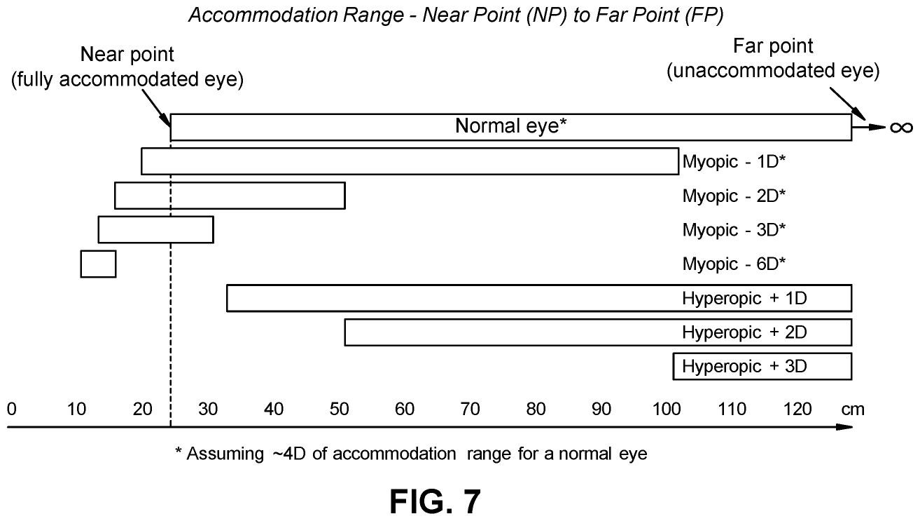

The physiology of the eye varies from person to person. For example, nearsightedness (myopia) and farsightedness (hyperopia—lifelong or presbyopia—age related) both affect the user's ability to visualize proper alignment. These issues are exacerbated when considering the utilization of the eye sprayer because its use prohibits the use of corrective lenses, as they would interfere with the delivery of the eye spray fluids. As a result, the user could have difficulty viewing the image of his/her eye in the mirror, which could prevent them from using the eye sprayer. When considering the accommodation range, i.e., the range within which the eye sprayer can be used, the varying degrees of myopia/presbyopia within the population needs to be taken into account. This best visualized in FIG. 7.

As shown in FIG. 7, the normal eye has a wide accommodation range from NP to FP. Nearsightedness and farsightedness introduce narrowed accommodation ranges, with varying degrees of overlap. From this, it is recognized that accommodation ranges for affected users can be adjusted via two properties: the working distance (WD) and the magnification introduced by the curvature of the mirror. By selecting the working distance from within the range of distances supported by the nozzle, in combination with the magnification produced by the mirror, a wide range of eyes can be accommodated.

### Concave Mirror Curvature

The concave curvature of the mirror 20 will have the effect of magnifying the image of the eye 70. Adjusting the curvature of the mirror thus changes its magnification while, at the same time, changing its focal length. While the working distance is limited by the configuration of the nozzle 30, it is a range, not a fixed distance, and can be taken into account when designing the mirror.

Accordingly, for the eye sprayer 10 disclosed herein, the curvature of the concave mirror 20 is advantageously configured to as to accommodate as large a portion of the population as possible. This is implemented by utilizing an optical ray tracing analysis to choose the optimal curvature based upon world population statistics for both nearsighted and farsighted people.

Through the implementation of an optical model, simulated conditions/results are determined in order to evaluate the efficacy of a particular mirror configuration. The radius of curvature (ROC) of the mirror and the working distance of the mirror are evaluated to determine their effects on users with varying degrees of myopia/presbyopia by simulating what is projected onto the retina. A wide variety of working distance and mirror ROC were simulated to determine the best combinations.

The mirror ROC is chosen so that the user with a given prescription can view the magnified virtual image of their own eye on the mirror 20 with the eye sprayer 10 positioned at a distance comfortable for the user's prescription. The eye sprayer can thereby accommodate users with a wide range of prescriptions. The precise value of the mirror ROC depends on the target eye sprayer working distance range. For example, for a working distance of 25 mm, a mirror ROC of 55±2 mm produces a resulting magnification of ˜8.8×, which can accommodate a range of eyes from myopia −6D to hyperopia +3D. A longer working distance results in a larger optimal ROC and lower magnification. Still, for a working distance of 30 mm, a mirror ROC of 67±2 mm produces a resulting magnification of ˜7.3×, which can also accommodate a range of eyes from myopia −6D to hyperopia +3D. Additionally, the simulation shows the amount of image blur that will be present for the above parameters for users with varying prescriptions at these working distances is not so detrimental as to prevent use of the eye sprayer 10. Nevertheless, the eye sprayer 10 can be configured so that the mirror 20 is interchangeable, with a variety of mirrors configured for different corrective lens prescription ratings being supplied. The user can select and install the mirror 20 best suited for his/her prescription. The eye sprayer 10 can there fore be optimized for that particular user.

Because the eye image is highly magnified by the mirror 20, even in the presence of image blur, alignment can be simple and intuitive. In fact, usability for more severe cases of myopia/hyperopia outside the ranges shown above can be accommodated. Thus, a user with myopia of −7D or worse or one with hyperopia of +4d or worse can be accommodated. In other words, despite the increased image blurriness for these more severe cases, the user can still make out proper alignment and use the eye sprayer effectively.

From the above description, those skilled in the art will perceive improvements, changes, and modifications. These and other such improvements, changes and modifications within the skill of the art are intended to be covered by the appended claims.

We claim:

1. An eye sprayer comprising:
a spray nozzle having a nozzle axis, the spray nozzle being configured to produce eye spray;
a concave mirror with an opening configured to allow for the delivery eye spray from the spray nozzle; and
a light emitter configured to produce a visible light beam directed from the mirror along the nozzle axis.

2. The eye sprayer recited in claim 1, wherein the opening in the mirror comprises a series of slits, and the spray nozzle comprises a series of nozzles, with a nozzle being positioned centrally in each slit, and wherein the light emitter is configured to produce a light beam emitted through each slit.

3. The eye sprayer recited in claim 1, wherein the eye spray comprises at least one of drops, a mist, and a micro-sheet of eye drop fluid.

4. The eye sprayer recited in claim 1, wherein a central axis of the mirror and the nozzle axis are coincident and parallel.

5. The eye sprayer recited in claim 1, wherein the light emitter is configured to direct the light through the spray nozzle along the nozzle axis.

6. The eye sprayer recited in claim 1, wherein the eye sprayer is configured to allow the user to view his/her eye at a prescribed working distance, and align the spray nozzle by aligning the image of their own eye with a view of the light source overlaid on top of it.

7. The eye sprayer recited in claim 6, wherein the mirror has a radius of curvature (ROC) configured to produce a degree of magnification that allows users with myopia or hyperopia/presbyopia to view his/her eye with the eye sprayer positioned at a prescribed working distance.

8. The eye sprayer recited in claim 7, wherein the mirror has a radius of curvature of 55±2 mm, a working distance of 25 mm.

9. The eye sprayer recited in claim 7, wherein the mirror has a radius of curvature of 67±2 mm, a working distance of 30 mm.

10. The eye sprayer recited in claim 7, wherein the mirror is an interchangeable insert, and its radius of curvature is chosen to allow optimal focus position based on the user eye prescription.

11. The eye sprayer recited in claim 7, wherein the mirror has a radius of curvature configured to allow the user to view his/her eye with sufficient clarity and lack of blurriness to allow them to align the view his/her eye with the eye sprayer positioned at a prescribed working distance.

12. The eye sprayer recited in claim 11, wherein the mirror is configured to accommodate users with myopia of at least −6D or hyperopia/presbyopia of at least +3D.

13. The eye sprayer recited in claim 1, wherein the mirror is semi-transparent and allows viewing additional light sources located behind the mirror to aid alignment.

14. The eye sprayer recited in claim 13, wherein the mirror has a semi-transparency of about 30% transmission about 70% reflectivity.

15. The eye sprayer recited in claim 13, wherein the light emitter comprises a light ring concentric with the mirror and extending around the nozzle.

16. The eye sprayer recited in claim 1, further comprising a proximity sensor configured to determine the proximity of the sprayer to the eye and to produce a visual indication of the sprayer being inside or outside a prescribed range of working distances.

17. The eye sprayer recited in claim 16, wherein the mirror is semi-transparent and the proximity sensor detects the proximity of the sprayer to the eye through the mirror.

18. The eye sprayer recited in claim 16, wherein the proximity sensor comprises an ultrasonic proximity sensor, a light-based position sensor, or a camera-based position sensor.

19. The eye sprayer recited in claim 1, wherein the opening in the mirror comprises a series of slits, and wherein the light emitter is configured to produce a light beam emitted through each slit.

20. The eye sprayer recited in claim 19, wherein the spray nozzle comprises a series of nozzles, with a nozzle being positioned centrally in each slit.
